(19) United States
(12) Patent Application Publication
(10) Pub. No.: US 2013/0217034 A1
(43) Pub. Date: Aug. 22, 2013

(54) PROCESS FOR AVOIDING FALSE POSITIVE RESULTS IN A DETECTING PROCESS OF AN INFLAMMATION INDICATOR IN A RINSE SOLUTION FOR TAKING UP GINGIVAL CREVICULAR FLUID

(71) Applicants: Kurt Maier, Berlin (DE); Timo Sorsa, Helsinki (FI)

(72) Inventors: Kurt Maier, Berlin (DE); Timo Sorsa, Helsinki (FI)

(73) Assignee: DENTOGNOSTICS GMBH, Jena (DE)

(21) Appl. No.: 13/816,696

(22) PCT Filed: Aug. 12, 2011
(86) PCT No.: PCT/EP2011/063931

§ 371 Date: Apr. 15, 2013

Related U.S. Application Data
(60) Provisional application No. 61/373,396, filed on Aug. 13, 2010.

(30) Foreign Application Priority Data
Aug. 13, 2010 (EP) 10172774.1
Jul. 7, 2011 (EP) 11173105.5

(51) Int. Cl. G01N 33/573

(52) U.S. Cl. CPC G01N 33/573
USPC 435/7.4; 435/283.1

(57) ABSTRACT

A process for avoiding false positive results in a detecting process of an inflammation indicator from the matrix metalloproteinase (MMP) family in a gingival crevicular fluid (GCF), wherein said GCF, which is obtained from a mouthrinse or saliva, is filtered before said inflammation indicator from the MMP family is assayed.

The present invention relates to a process for avoiding false positive results in a detecting process of an inflammation indicator from the matrix metalloproteinase (MMP) family in a gingival crevicular fluid (GCF) taken up in a mouthrinse, and to the use of filters in the process according to the invention.

One of the most wide-spread chronic diseases is inflammation of the parodontium. Actually, more teeth are lost because of parodontal diseases than because of dental caries. Therefore, there is a great need for reliable tests for the diagnosis of parodontal diseases. The parodontal diseases include a group of inflammatory diseases caused by infections afflicting the gingiva (gum), the parodontium (Sharpey fibers) and the tooth-bearing alveolar bone structures (of the jaw). The primary cause of parodontal diseases is bacterial plaques (soft and hard plaques) adhering to the tooth. This causes inflammations of the gingiva, which may consequently lead to the destruction of the actual parodontium by an overreaction of the endogenous immune defense. In parodontal diseases, a high accumulation of bacteria is usually found in the plaques, both above (supragingival) and below (subgingival) the gingival margin. These plaques accommodate, among others, so-called parodontally pathogenic germs including anaerobic germs. Consequently, the host organism prepares the surrounding tissue structures for an immunological defense by means of inflammation mediators. One of the known mechanisms is the release and activation of large amounts of collagenases (matrix metalloproteinases) by the polymorphonuclear (PMN) cells of the immune defense. In the literature, these collagenases are considered the chiefly responsible causes of the degradation of the parodontal tissue. So-called pockets may form between the tooth and the gingiva, which is an essential characteristic of parodontal diseases.

A gingivitis (inflammation of the gum) is distinguished from a periodontitis in that although the gingiva is inflamed in a gingivitis, deep (>4 mm) periodontal pockets are not yet detectable; therefore, a gingivitis is not accompanied at first by the irreversible destruction of tooth-bearing structures. A periodontitis is characterized by an inflamed gingiva and destruction of tooth-bearing structures; however, it may happen that a periodontitis is not recognized when the gingiva looks clinically healthy.

The purification of human neutrophil collagenase (MMP-8) was described by Yoshida and Naoki, Kokubyo Gakkai Zasshi, 60(1), 121-130, 1993 (Chemical Abstracts 119, 15, 1993, AN 154562x). Monoclonal antibodies against latent or non-activated neutrophil collagenase or MMP-8 were prepared. The monoclonal antibodies were used for staining neutrophil collagenase, which occurred in granules of neutrophils from gingival pockets. These monoclonal antibodies do not discriminate between active and latent MMP-8 and therefore cannot be employed in the diagnosis of parodontal disease activity.

EP-B-777 859 relates to the use of monoclonal antibodies which recognize the active form of MMP-8 from mammals and discriminate between active and proenzymatic forms and therefore offer a means for providing reliable and sensitive methods and test kits for the diagnosis of periodontitis. Antibodies employed for the detection of active MMP-8 are described. Further, a process is introduced for the reliable, repeatable, sensitive and specific diagnosis of parodontal disease activity based on monoclonal antibodies that recognize active mammal matrix metalloproteinase-8, in addition to those that can be employed in outpatient diagnosis and are not only specific, sensitive and reliable, but are also quickly and simply performed.

WO-A-2004/092733 discloses methods and compositions, e.g., to reduce interference from non-specific binding sample constituents in a migration shift assay. Interference due to non-specific binding of sample constituents to an affinity substance (e.g., an affinity molecule or a conjugate of an affinity molecule and a charged carrier molecule) is prevented by, e.g., binding the constituents to charged polymers such as heparin sulfate. The application also provides methods to concentrate an analyte of interest with high concentration and to detect the analyte with high sensitivity, and further to optimize the reaction conditions for easily concentrating the analyte. The objects of the application are attained by concentrating a complex of the analyte and a conjugate which is formed by contacting the analyte in a sample with an affinity molecule bound to a charged carrier molecule such as DNA.

US-A-2006127886 discloses a lateral flow assay device for detecting the presence or quantity of an analyte residing in a test sample where the lateral flow assay device has a porous membrane in communication with a conjugate pad and a wicking pad. The porous membrane has a detection zone which has an immobilized first capture reagent configured to bind to at least a portion of the analyte and analyte-conjugate complexes to generate a detection signal. A control zone may be located downstream from the detection zone on the porous membrane and has a second capture reagent immobilized within the control zone. The conjugate pad is located upstream from the detection zone, and has detection probes with specific binding members for the analyte. The sample is deposited between the control and detection zones. A buffer release zone is located upstream of the conjugate pad and provides for buffer addition to the device, the buffer serving to move the detection probes to the detection and control zones.

WO-A-00/42434 discloses a method and a device for the differential determination of a quantity of at least one analyte in a liquid sample. A test strip which has at least two areas is used, these areas being composed in such a way that they can provide information about the quantity of the at least one analyte. The test strip also ensures that the sample or the portion of the sample flows laterally through the strip and the at least two areas by having a predetermined capacity for binding the at least one analyte in the first area. When this capacity is exceeded by too great a quantity of the at least one analyte, the at least one analyte is bound in at least one second area, the capacity of the second area being essentially equal to the capacity of the first area and the binding of the at least one analyte in the at least one first area and the at least one second area being measured by a detectable property.

WO-A-90/10062 discloses methods and compositions for testing for various types of human cancers, including in particular metastatic mammary carcinoma, colon carcinoma astrocytoma, renal cell carcinoma, and teratocarcinoma malignant melanoma. The diagnostic involves the detection of a gelatinolytic and type IV collagenolytic metalloproteinase having molecular weight of approximately 88 to 92 KDa in size. This high molecular weight type IV collagenolytic enzyme is found to be associated with the cancerous state, but this has not been found in any noncancerous cells tested and further not found in the serum of non tumor bearing animals. The enzyme is associated both with rat and human tumors, the enzyme in each case being quite similar. Methods are disclosed for isolating the 88/92 kd enzyme, the isolated enzyme being useful as a control enzyme and diagnostic assays. Furthermore, the isolated enzyme will be useful in the preparation of antibodies, including monoclonal antibodies, having specificity for the metastasis-associated enzyme. Techniques are also disclosed for isolation and partial purification of the enzyme from both rat and human sources. Furthermore, methods are disclosed for conducting both enzymatic and immunologic based tests for the diagnosis of metastatic and recurrent disease.

US-A-2006/223193 discloses a diagnostic test kit that employs a lateral flow assay device and a plurality of assay reagents for detecting a test analyte within a test sample is disclosed. The assay reagents include detection probes that are capable of producing a detection signal representing the presence or quantity of the test analyte in the test sample. To further enhance detection accuracy, calibration probes are also used that are capable of producing a calibration signal representing the presence or quantity of a calibration analyte. The calibration signal may be utilized to calibrate the detection signal.

Balwant Rai et al. disclose in Journal of Oral Science, Vol. 50, No. 1, 53-56, 2008 biomarkers of periodontitis in oral fluids. It refers to a study which determined the levels of GCF matrix metalloproteinase-2 (MMP-2) and metalloproteinase-9 (MMP-9) and salivary MMP-8 in patients with gingivitis and periodontitis and in healthy controls. Significantly higher salivary MMP-8 and crevicular MMP-9 were observed in cases of periodontitis compared to gingivitis and healthy adults. On the other hand, crevicular MMP-2 levels in periodontitis subjects were lower than those in gingivitis and healthy subjects.

Surprisingly, it has been found that in the case of saliva specimen or rinses of the oral cavity, specimens that had been subjected to filtration prior to the application of the actual diagnostic method yielded useful or even just reliable results that allowed a clinical statement to be made about the patient's condition, avoiding or excluding false positive results.

The present invention relates to a process for avoiding false positive results in a detecting process of an inflammation indicator from the matrix metalloproteinase (MMP) family in a gingival crevicular fluid (GCF) taken up in a rinse or in saliva, wherein said GCF obtained from a mouthrinse or saliva is filtered before said inflammation indicator from the MMP family is assayed.

In particular, the process according to the invention can be regarded as a sample preparation process before a diagnostic process is performed. It is extremely robust and can be performed with simple methods “on the chair side”, i.e., directly near the patient in close temporal relation to the sampling, and can be subjected to a diagnostic test, for example, in a method per se known to the skilled person, such as a lateral flow test, dipstick test or other method.

The inflammation indicator is a matrix metalloproteinase (MMP), especially MMP-8. Especially advantageous is the detection of active matrix metalloproteinase, especially active MMP-8.

According to the invention, it has been found useful to employ filters selected from the group consisting of regenerated cellulose (RC), nitrocellulose (NC), cellulose nitrate (CN), cellulose acetate (CA), cellulose mixed ester (CME), polyamide (PA), polytetrafluoroethylene (PTFE), polyvinylidene fluoride (PVDF), polypropylene (PP), polyester (PE), polyethersulfone (PES), glass fiber mat (GFM), stainless steel. The filters may have an average pore size of about 0.1, 0.2, 0.45, 0.8, 1.2, 5.0 μm.

The inflammation indicator to be specifically detected by the process according to the invention, for example, in a lateral flow test or a dipstick test, is indicative of periodontitis and periimplantitis.

In particular, the diagnostic detection method is a detection method based on antibody assays, typically a strip test (lateral flow test, dipstick test), enzyme immunoassay (EIA), enzyme-linked immunosorbent assay (ELISA), immunofluorescence marked assay (IFMA), Western blot, antibody immunocolumn for analytical processes (ABICAP) test method.

The invention also relates to the use of a filter selected from the group consisting of PES, PA, RC, PDVF, CN, NC, CME, especially with an average pore size of 0.2, 0.45, 0.8 μm. WO-A-2009/118423 describes in detail an analytical device typically to be employed for diagnostics, and a related process. The whole disclosed content of this specification is included herein by reference.

### EXAMPLE

The patient rinses his or her oral cavity with tap water for about 30 seconds and spits the water out or swallows it. After a waiting time of approximately 1 minute, the rinsing solution, which consists of water (aqua purificata with or without additives, such as flavors, salts), is given to the patient for rinsing. He or she rinses his or her mouth, teeth and interdental spaces intensively with the rinsing solution for 30 seconds. The volume of the rinsing solution may be about from 3 to 10 ml. A volume of about 5 ml has been found to be ideal. Gurgling should be avoided. The patient will return the mouthrinse as completely as possible into a cup or vessel, for example, the one from which the rinsing solution was taken, wherein the amount of liquid should not be significantly smaller than the amount taken up. This sample is drawn into a syringe, which is connected with a commercially available filter unit (so-called syringe filter). From the syringe and through the filter unit, the amount of sample solution required for the subsequent test method, for example, 3 drops, is delivered onto a lateral flow test. After the application of the sample, the test strip is read after a time according to the test method, for example, after about 5 minutes for a lateral flow test, and an increased risk for a parodontal tissue degradation can be read from the test strip accordingly if two lines (test line and control line) are stained.

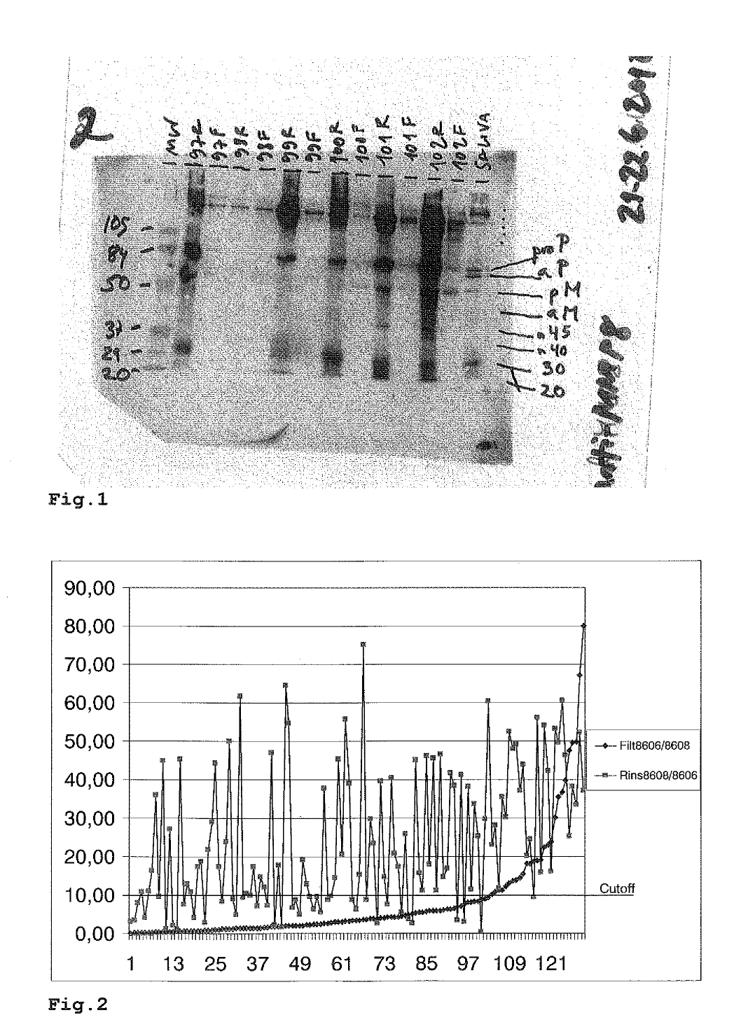

FIG. 1 shows a Western blot comparison of samples 97 to 102, in each case comparing the original mouthrinse (R) and the related filtrate of the mouthrinse (F).

It becomes clear that the complexes occurring above 105 kD (see the scale on the left and the MW calibrator in the first column of the blot) in the original rinses designated with R are significantly reduced in the respective adjacent blots of the related filtrates. Such complexes lead to disturbances and false positive signals in an assay.

FIG. 2 shows the total of 130 examined specimens in ascending sorting by the MMP concentration in the filtrate (-⋄-Filt8606/8608) and the respectively related MMP concentration in the original unfiltered rinse (-570 -Rins8606/8608). The filtrates reflect the clinically expected values.

1. A process for avoiding false positive results in a detecting process of an inflammation indicator from the matrix metalloproteinase (MMP) family in a gingival crevicular fluid (GCF), said process comprising filtering said GCF, which is obtained from a mouthrinse or saliva, before said inflammation indicator from the MMP family is assayed.

2. The process according to claim 1, characterized in that said matrix metalloproteinase is an active matrix metalloproteinase.

3. The process according to claim 1, characterized in that said filter is selected from the group consisting of regenerated cellulose (RC), nitrocellulose (NC), cellulose nitrate (CN), cellulose acetate (CA), cellulose mixed ester (CME), polyamide (PA), polytetrafluoroethylene (PTFE), polyvinylidene fluoride (PVDF), polypropylene (PP), polyester (PE), polyethersulfone (PES), glass fiber mat (GFM), and stainless steel.

4. The process according to claim 3, characterized in that said filter has an average pore size in a range of about 0.1 to about 5.0 μm.

5. The process according to claim 1 further comprising a diagnosis that said inflammation indicator is indicative of periodontitis and periimplantitis.

6. The process according to claim 1, characterized in that the detection method is a detection method based on antibody assays.

7. The process according to claim 6, characterized in that said detection method based on antibody assays is chosen from the group consisting of a strip test (lateral flow test, dipstick test), enzyme immunoassay (EIA), enzyme-linked immunosorbent assay (ELISA), immunofluorescence marked assay (IFMA), Western blot, and antibody immunocolumn for analytical processes (ABICAP) test method.

8. The process of claim 1 comprising performing the filtering with a filter selected from the group consisting of PES, PA, RC, PDVF, CN, NC, CME in a process according to at least one of the preceding claims.

9. The process of to claim 8, characterized in that said filter has an average pore size in a range from 0.2 to 0.8 μm.

10. A detection kit for detecting an inflammation indicator from the matrix metalloproteinase (MMP) family in a gingival crevicular fluid (GCF), comprising a filter for GCF.

11. The kit of claim 10 wherein the filter is selected from the group consisting of regenerated cellulose (RC), nitrocellulose (NC), cellulose nitrate (CN), cellulose acetate (CA), cellulose mixed ester (CME), polyamide (PA), polytetrafluoroethylene (PTFE), polyvinylidene fluoride (PVDF), polypropylene (PP), polyester (PE), polyethersulfone (PES), glass fiber mat (GFM), and stainless steel.

12. The kit of claim 10 wherein the filter is a syringe filter.
